(19) United States
(12) Patent Application Publication
(10) Pub. No.: US 2008/0182890 A1
(43) Pub. Date: Jul. 31, 2008

(54) METHODS OF USING LOW-DOSE DOXEPIN FOR THE IMPROVEMENT OF SLEEP

(75) Inventors: Philip Jochelson, San Diego, CA (US); Roberta L. Rogowski, Rancho Santa Fe, CA (US)

Correspondence Address: KNOBBE MARTENS OLSON & BEAR LLP, 2040 MAIN STREET, FOURTEENTH FLOOR, IRVINE, CA, 92614, US

(73) Assignee: SOMAXON PHARMACEUTICALS, INC., San Diego, CA (US)

(21) Appl. No.: 11/867,595

(22) Filed: Oct. 4, 2007

Related U.S. Application Data
(60) Provisional application No. 60/849,644, filed on Oct. 4, 2006.
(60) Provisional application No. 60/943,503, filed on Jun. 12, 2007.

(51) Int. Cl. A61K 31/336; A61P 43/00

(52) U.S. Cl. 514/450

(57) ABSTRACT

Methods of treating sleep disorders by administration of low doses of doxepin in individuals seeking sustained efficacy or in need of avoiding weight gain, rebound insomnia, or sedative tolerance resulting from doxepin treatment.

### CROSS-REFERENCE TO RELATED APPLICATIONS

This application claims priority to U.S. Provisional Application No. 60/849,644 filed on Oct. 4, 2006 and U.S. Provisional Application No. 60/943,503 filed on Jun. 12, 2007, both entitled METHODS OF USING LOW-DOSE DOXEPIN FOR THE IMPROVEMENT OF SLEEP; and each of which is incorporated herein by reference in its entirety.

### FIELD OF THE INVENTION

The present invention relates to the use of doxepin to treat individuals with sleep disorders who are also in need of avoiding weight gain, rebound insomnia, or sedative tolerance resulting from pharmacological treatment of the disorder. BACKGROUND OF THE INVENTION

Sleep is essential for health and quality of life. Insomnia is a growing health problem in the United States. It is believed that more than 30-45 million people suffer from chronic insomnia and up to an additional 70 million people suffer from some form of insomnia each year. Insomnia is a condition characterized by difficulty falling asleep (sleep onset), waking frequently during the night (fragmented sleep), waking too early (premature final awakening), and/or waking up feeling un-refreshed. In the National Sleep Foundation's (NSF) Sleep in America Poll 2005, 42% of survey respondents reported that they awoke frequently during the night, 22% of adults reported waking too early and not being able to return to sleep and 38% reported waking and feeling un-refreshed.

Sleep maintenance difficulty is the most commonly reported symptom in primary care patients with chronic insomnia, and is the most common insomnia complaint in depressed patients, medically ill populations, especially those with pain symptoms, and in the elderly.

Medications commonly used to treat sleep disorders, such as insomnia, include sedative antidepressants, antihistamines, antipsychotics, benzodiazepines, and non-benzodiazepine hypnotics. Unfortunately, many of these existing medications have undesirable side effects such as weight gain or result in rebound insomnia or sedative tolerance.

The present invention describes the surprising ability of doxepin to treat insomnia, without the untoward side effects of unwanted weight gain, rebound insomnia, or sedative tolerance.

### SUMMARY OF THE INVENTION

Some embodiments provide a method for selecting a drug to be administered to a patient in need of sleep therapy, comprising evaluating the importance of avoiding weight gain for that particular patient in conjunction with the sleep therapy; and selecting doxepin therapy for the patient if avoiding weight gain is evaluated to be of sufficient importance. In some aspects the doxepin therapy can be a therapy that utilizes doxepin, as well as prodrugs and pharmaceutically acceptable salts of doxepin.

Other embodiments provide a method for selecting a drug to be administered to a patient in need of sleep therapy, comprising evaluating the importance of avoiding rebound insomnia for that particular patient in conjunction with the sleep therapy; and selecting doxepin therapy for the patient if avoiding rebound insomnia is evaluated to be of sufficient importance. In some aspects the doxepin therapy can be a therapy that utilizes doxepin, as well as prodrugs and pharmaceutically acceptable salts of doxepin. In some preferred aspects, the patient can be one that is suffering from a short term or transient insomnia.

Still other embodiments provide a method for selecting a drug to be administered to a patient in need of sleep therapy, comprising evaluating the importance of avoiding sedative tolerance for that particular patient in conjunction with the sleep therapy; and selecting doxepin therapy for the patient if avoiding sedative tolerance is evaluated to be of sufficient importance. In some aspects the doxepin therapy can be a therapy that utilizes doxepin, as well as prodrugs and pharmaceutically acceptable salts of doxepin. In some preferred aspects, the patient can be one that is suffering from a chronic insomnia.

Yet another embodiment provides a method for selecting a drug to be administered to a patient in need of sleep therapy, comprising evaluating the importance of maintaining a sustained efficacy over a period of long-term use; and selecting doxepin therapy for the patient if maintaining sustained efficacy over a period of long-term use is evaluated to be of sufficient importance. In some aspects the doxepin therapy can be a therapy that utilizes doxepin, as well as prodrugs and pharmaceutically acceptable salts of doxepin. In some preferred aspects, the patient can be one that is suffering from a short term or transient insomnia.

In some aspects, the method may further comprise administering a daily dosage of doxepin in an amount of about 0.5 milligrams to about 6 milligrams. The administering a daily dosage of doxepin also can include administering a pharmaceutically acceptable salt or a prodrug of doxepin. In any of the above mentioned embodiments, the patient can be in need of treatment of a transient insomnia, a short term insomnia or a chronic insomnia, for example.

Some embodiments provide a method comprising identifying a patient in need of pharmaceutical treatment of a sleep disorder while at the same time avoiding weight gain resulting from the treatment; and treating the sleep disorder in the patient by administering doxepin, a as well as prodrugs and pharmaceutically acceptable salts of doxepin in a dosage between about 0.5 milligram and 6 milligrams.

Yet other embodiments provide a method comprising identifying a patient in need of pharmaceutical treatment of a sleep disorder while at the same time avoiding rebound insomnia resulting from the treatment; and treating the sleep disorder in the patient by administering doxepin, as well as prodrugs and pharmaceutically acceptable salts of doxepin in a dosage between about 0.5 milligram and 6 milligrams. In some preferred aspects, the patient can be one that is in need of treatment of a short term or transient insomnia.

Further embodiments provide a method comprising identifying a patient in need of pharmaceutical treatment of a sleep disorder while at the same time avoiding sedative tolerance resulting from the treatment; and treating the sleep disorder in the patient by administering doxepin, as well as prodrugs and pharmaceutically acceptable salts of doxepin in a dosage between about 0.5 milligram and 6 milligrams. In some preferred aspects, the patient can be one that is in need of treatment of a chronic insomnia.

Still further embodiments provide a method comprising identifying a patient in need of pharmaceutical treatment of a sleep disorder while at the same time maintaining a sustained efficacy of the treatment during a period of long-term use of the pharmaceutical treatment; and treating the sleep disorder in the patient by administering doxepin, as well as prodrugs and pharmaceutically acceptable salts of doxepin in a dosage between about 0.5 milligram and 6 milligrams. In some preferred aspects, the patient can be one that is in need of treatment of a chronic insomnia.

In other embodiments provided are methods comprising identifying a patient in need of pharmaceutical treatment of a sleep disorder and also susceptible to weight gain resulting from treatment of the sleep disorder using a medication other than doxepin; and treating the sleep disorder in the patient by administering doxepin, as well as prodrugs and pharmaceutically acceptable salts of doxepin in a dosage between about 0.5 milligram and 6 milligrams. In some aspects, the patient may be considered susceptible due to having experienced weight gain while taking a different sleep medication.

In other embodiments provided are methods comprising identifying a patient in need of pharmaceutical treatment of a sleep disorder and also susceptible to rebound insomnia resulting from treatment of the sleep disorder using a medication other than doxepin; and treating the sleep disorder in the patient by administering doxepin, as well as prodrugs and pharmaceutically acceptable salts of doxepin in a dosage between about 0.5 milligram and 6 milligrams. In some aspects, the patient may be considered susceptible due to having experienced rebound while taking a different sleep medication. In some preferred aspects, the patient can be one that is in need of treatment of a short term or transient insomnia.

In other embodiments provided are methods comprising identifying a patient in need of pharmaceutical treatment of a sleep disorder and also susceptible to sedative tolerance resulting from treatment of the sleep disorder using a medication other than doxepin; and treating the sleep disorder in the patient by administering doxepin, as well as prodrugs and pharmaceutically acceptable salts of doxepin in a dosage between about 0.5 milligram and 6 milligrams. In some aspects, the patient may be considered susceptible due to having experienced sedative tolerance while taking a different sleep medication. In some preferred aspects, the patient can be one that is in need of treatment of a chronic insomnia.

In other embodiments are provided a method comprising identifying a patient in need of pharmaceutical treatment of a sleep disorder and also susceptible to reduced pharmaceutical efficacy over a period of long-term use of a medication other than doxepin; and treating the sleep disorder in the patient by administering doxepin, as well as prodrugs and pharmaceutically acceptable salts of doxepin in a dosage between about 0.5 milligram and 6 milligrams. In some aspects, the patient may be considered susceptible due to having experienced reduced efficacy with a different sleep medication. In some preferred aspects, the patient can be one that is in need of treatment of a chronic insomnia.

In some aspects, the dosage of doxepin can be about 0.5 milligrams, 1 milligram, about 3 milligrams or about 6 milligrams. In some aspects the dosage of doxepin is about 0.5 milligrams. In some aspects, the dosage of doxepin is about 1 milligram. In some aspects, the dosage of doxepin is about 3 milligrams. In still other embodiments, the dosage of doxepin is about 6 milligrams.

In any of the above mentioned embodiments, the patient can be in need of treatment of a transient insomnia, a short term insomnia or a chronic insomnia, for example.

Still further embodiments relate to methods of providing a sleep therapy, which methods can include informing a medical caregiver or a patient that doxepin in an amount of about 1 to 6 mg does not result in weight gain or in significant weight gain; and providing the caregiver or the patient with about 1-6 mg of doxepin for consumption by the patient. In some aspects the information can be provided to either the caregiver or to the patient, or to both.

Also, some embodiments relate to methods of providing a sleep therapy, which methods can include informing a medical caregiver or a patient that doxepin in an amount of about 1 to 6 mg does not result in sedative tolerance or in significant sedative tolerance; and providing the caregiver or the patient with about 1-6 mg of doxepin for consumption by the patient. In some aspects the information can be provided to either the caregiver or to the patient, or to both. In some preferred embodiments the patient can be in need of treatment for a chronic insomnia, for example.

Some embodiments relate to methods of providing a sleep therapy, which methods can include informing a medical caregiver or a patient that doxepin in an amount of about 1 to 6 mg does not result in rebound insomnia or in significant rebound insomnia; and providing the caregiver or the patient with about 1-6 mg of doxepin for consumption by the patient. In some aspects the information can be provided to either the caregiver or to the patient, or to both. In some preferred embodiments the patient can be in need of treatment for a short term or transient insomnia, for example.

Further embodiments relate to methods of providing a sleep therapy, which methods can include informing a medical caregiver or a patient that doxepin in an amount of about 1 to 6 mg maintains sustained efficacy or significantly sustained efficacy as a sleep therapeutic over a period of long term use or chronic use; and providing the caregiver or the patient with about 1-6 mg of doxepin for consumption by the patient. In some aspects the information can be provided to either the caregiver or to the patient, or to both. In some preferred embodiments the patient can be in need of treatment for a chronic insomnia, for example.

Also, some embodiments relate to doxepin treatments of a sleep disorder in a patient, wherein the patient is in need of avoiding one or more of rebound insomnia, reduced efficacy, sedative tolerance or weight gain. In some aspects the patient may have experienced one or more of the listed adverse effects while taking a non-doxepin sleep medication. In some aspects the patient can be in need of treatment of a transient, short term or chronic insomnia, for example. The dosage of doxepin can be, for example, between about 0.5 and 6 mg, about 0.5 milligrams, about 1 milligram, about 3 milligrams or about 6 milligrams, for example.

Some embodiments relate to doxepin for the treatment of a transient or short term sleep disorder in a patient, wherein the patient is in need of avoiding or minimizing rebound insomnia. Other embodiments relate to doxepin for the treatment of a chronic sleep disorder in a patient, wherein the patient is in need of avoiding or minimizing sedative tolerance. The dosage of doxepin can be, for example, between about 0.5 and 6 mg, about 0.5 milligrams, about 1 milligram, about 3 milligrams or about 6 milligrams, for example.

In any of the above mentioned embodiments, the patient can be in need of treatment of a transient insomnia, a short term insomnia or a chronic insomnia, for example.

### BRIEF DESCRIPTION OF THE DRAWINGS

FIG. 1 is a graph showing sustained sleep improvement as measured by polysomnography (PSG) efficacy variables: latency to persistent sleep (LPS), wake time after sleep onset (WASO) and total sleep time (TST). Data for 3 mg and 6 mg treatment groups is shown as mean change (in minutes) compared to baseline, for night 1 (N1) and night 29 (N29). P-values of p<0.05 versus placebo are indicated by an asterisk.

FIG. 2 is a graph showing sustained sleep improvement as measured by subjective variables: latency to sleep onset (LSO), subjective WASO (sWASO) and subjective TST (sTST). Data for 3 mg and 6 mg treatment groups is shown as mean change (in minutes) compared to baseline, for night 1 (N1) and night 29 (N29). P-values of p<0.05 versus placebo are indicated by an asterisk.

FIG. 3 is a graph showing that no rebound insomnia occurred during the 2-day discontinuation period following abrupt discontinuation of 35 nights of treatment. Results for the 3 mg and 6 mg doxepin groups are presented as WASO (in minutes) at baseline and night 29 compared to the discontinuation period at nights 36 and 37.

FIG. 4 is a graph showing that no rebound insomnia occurred during the 2-day discontinuation period following abrupt discontinuation of 35 nights of treatment. Results for the 3 mg and 6 mg doxepin groups are presented as WASO (in minutes) at baseline and treatment night 29 compared to nights 1 and 2 after doxepin discontinuation.

### DETAILED DESCRIPTION OF THE INVENTION

Sleep disorders are a growing health problem. However, many of the medications that are currently used to treat the disorders result in untoward side effects. The present invention generally relates to methods of using doxepin, prodrugs and pharmaceutically acceptable salts of the same, to treat a sleep disorder, such as insomnia, while avoiding or minimizing the side effects of weight gain, sedative tolerance or rebound insomnia. Also, some embodiments relate to methods for selecting a drug to be administered to a patient in need of sleep therapy. The methods are described more fully below. The methods relate to the unexpected finding that using doxepin to improve sleep of an individual does not result in weight gain, sedative tolerance, or rebound insomnia.

Weight gain is a common problem associated with the use of most sleeping disorder drugs. Tricyclic antidepressants (TCAs) are well known for their effect on weight, both in the short and long term. Weight gain is a common adverse event after treatment with TCAs, even for 1 month. This is supported by studies showing that antidepressants in general, and tricyclics in particular, are associated with a risk of weight gain. See, for example, Fava, J. Clin. Psychiatry. (2000) 61:37-41; Claudino et al., Cochrane Database Syst Rev. (2006) 1:1-39; Laimer et al., J. Clin. Psychiatry (2006) 421-424; each of which is incorporated by reference in its entirety. Recent reports in the popular press have linked zolpidem (commonly known as Ambien™) to unconscious binge eating and weight gain. See, for example, Stephanie Saul, NY Times, Mar. 14, 2006, and CBS News Early Show, Mar. 15, 2006 (at the hypertext transfer protocol on the world wide web at CBS.com). Weight gain is an infrequent, but known adverse effect of benzodiazepine and non-benzodiazepine GABA receptor antagonists. See, for example, Physicians Desk Reference 2006 monographs for Prosom™ (estazolam) and Sonata™ (zaleplon). Antipsychotic drugs such as Zyprexa™ (olanzapine), used to treat insomnia in some populations, have been strongly associated with weight gain. See, for example, Newcomer et al., “The metabolic effects of antipsychotic medications,” Can J Psychiatry. 2006 July; 51 (8): 480-91. Each of the references mentioned in this paragraph is incorporated by reference in its entirety.

Similarly, relationships between dietary intake and central nervous system histaminergic activity have been suggested. Blockade of histamine receptors has been associated with weight gain. Histamine receptors have been implicated in the regulation of food and water intake in rodents (Masaki and Yshimatsu, Trends Pharmacol Sci 27: 2006). H₁ receptors located in the paraventricular nucleus in particular may be responsible for circadian patterns of feeding that may contribute to obesity (Masaki et al., Diabetes 53: 2004). H₁-deficient mice display increased food intake and visceral adiposity in conjunction with a disturbed circadian feeding rhythm. Accordingly, animals administered H₁ antagonists consume more food than controls and gain weight at a faster rate. The appetite stimulating effects of high H₁ affinity antidepressants in rats can be attenuated by compounds that deplete intracellular histamine (Ookuma et al., Psychopharmacology (1990) 101:481-485). Rats given low protein diets show a decrease in brain histamine and a decrease in food intake (Mercer et al., J. Am. Coll. Nutr. (1996) 15:223-230). Clinically, antidepressants and antipsychotics known to increase appetite tend to have high affinities for the H₁ receptor, and those that do not stimulate appetite tend to have low affinity for the H₁ receptor (Orthen-Gambill, Pharmacol Biochem Behav 31:1988; Orthen-Gambill and Salomon, Pharmacol Biochem Behav 36:1990). Each of the references mentioned in this paragraph is incorporated by reference in its entirety. Doxepin is known to be a very potent H₁ antagonist. See, for example, Richelson et al., J. Pharmacol. And Exper. Therapeutics, 230:94-102 (1984). In view of that, it is very surprising and unexpected that the low dose doxepin when used to treat sleep as described herein does not result in weight gain in patients.

Rebound insomnia is a common problem associated with hypnotic medications used to treat insomnia. In general, both the benzodiazepines and the non-benzodiazepine hypnotics are associated with rebound insomnia, with no clear evidence of superiority for either type. See, for example, Roehrs et al., in Kryger et al., eds., Principles and Practice of Sleep Medicine (2000) 414-8; Voshaar et al., Eur Neuropsychopharmacol (2004) 14:301-6; and Roth et al., Sleep Medicine (2006) 7:397-406. Additionally, it appears that the potential for rebound insomnia is greater in hypnotics with the shortest half-life. Rebound insomnia has also been associated with the discontinuation of tricyclic antidepressant agents when used to treat insomnia, although this phenomenon has not been studied extensively. See, for example, Hohagen et al., Eur Arch Psychiatry Clin Neurosci (1994) 244:65-72. Most symptoms related to tricyclic antidepressant withdrawal are believed to be caused by rebound excess of cholinergic activity after prolonged anticholinergic effect on cholinergic receptors. See, for example, Wolfe et al., Am. Fam. Physician (1997) 56:455-62. Each of the references mentioned in this paragraph is incorporated by reference in its entirety.

Development of tolerance to the sedative effects of antihistamines has frequently been described. See, for example, Richardson et al., J. Clin. Psychiatry (2002) 22:511-515; Manning et al., J. Clin. Psychiatry (1992) 32:996-1002; Schweitzer et al., J Allergy Clin. Immunol. (1994) 94:716-724; each of which is incorporated by reference in its entirety. Thus, there is a need for methods of treatment of sleep disorders that maintain sustained drug efficacy over a sustained period of time, for example during long term or chronic use. Doxepin satisfies the long-felt need for treatments of sleep disorders with sustained efficacy or a lack of development of tolerance. As mentioned above, doxepin is a strong H₁ antagonist (see Richelson et al. mentioned above). In view of that, it is very surprising and unexpected that drug efficacy of doxepin is sustained and that there is a lack of development of tolerance when using the methods described herein.

Doxepin is a tricyclic compound approved for the treatment of depression and anxiety.

The recommended daily dose for the treatment of depression and anxiety ranges from 75 milligrams to 300 milligrams. It is very surprising that doxepin is effective for treating sleep disorders without the above-mentioned adverse effects that are associated with other medications used to treat sleep disorders.

Doxepin, unlike most FDA approved products for the treatment of insomnia, is not a Schedule IV controlled substance. U.S. Pat. Nos. 5,502,047 and 6,211,229, the entire contents of which are incorporated herein by reference, describe the use of doxepin for the treatment chronic and non-chronic (e.g., transient/short term) insomnias at dosages far below those used to treat depression.

Surprisingly, as discussed more fully below, in a phase III study to assess the efficacy and safety of doxepin for the treatment of insomnia, little or no weight gain, rebound insomnia or sedative tolerance was observed. Instead, sustained efficacy was achieved over a period of long-term use.

Thus, some embodiments relate to methods of using doxepin, pharmaceutically acceptable salts of doxepin or prodrugs of doxepin to treat a patient suffering from a sleep disorder, while minimizing or avoiding weight gain, rebound insomnia, sedative tolerance, or while achieving sustained efficacy over periods of long-term use.

### Definitions

The term “insomnia” generally refers to sleep problems characterized by difficulty falling asleep, awakenings during the night, or waking up earlier than desired. Examples of insomnia include chronic and non-chronic insomnias. Transient and short term insomnia are examples of non-chronic insomnias. Also, sleep onset insomnia and sleep maintenance insomnia are examples of insomnia conditions that can be chronic or non-chronic in nature and duration.

As used herein, the term “transient insomnia” is an insomnia that is present for one to several days, and is less than one week in duration, Short term insomnia is insomnia of one (1) to three (3) or four (4) weeks in duration.

“Chronic insomnia” is typically accepted to involve episodes greater than three (3) or four (4) weeks in duration.

“Onset insomnia” refers to difficulty in falling asleep.

“Maintenance insomnia” refers to difficulty in maintaining uninterrupted sleep.

It is well known that the sleep deprivation resulting from such insomnia adversely affects cognition, safety and quality of life. Even in otherwise healthy young people, sleep deprivation has been associated, for example, with changes in body physiology such as changes in thyroid function, changes in glucose metabolism and insulin resistance.

The term “weight gain” refers to an increase in body weight. An increase in body weight can include without limitation, for example, an increase in weight compared to a baseline number or an increase when measured over a period of time. For example, the baseline number can be the weight of a patient prior to beginning a doxepin therapy or a weight taken during the therapy. Also, for example, the time period can be the period of time during which the patient is taking the doxepin therapy.

As used herein, the term “sedative tolerance” refers to a decreased response to a repeated drug dose which requires increasing amounts of the drug to produce the same effect. Tolerance is usually manifested by a decreased duration or magnitude of sedation in response to a given drug dose. Thus, the patient requires larger doses to produce the sedative effects. In contrast, sedative drugs which avoid sedative tolerance achieve sustained efficacy over periods of prolonged or long term use, for example during treatment of chronic sleep disorders.

The term “rebound insomnia” refers to the common occurrence that a person may have more trouble sleeping the first few nights after a sedative medicine is stopped than before starting the medicine. After more than a few days' use, discontinuing a hypnotic or sedative medication can make the original sleep problem worse and can cause an increase anxiety. Rebound insomnia may be measured as the mean change in mean wake time after sleep onset (WASO) from baseline to the first night of discontinuation.

As used herein, the term “polysomnography” (PSG) refers to a diagnostic test during which a number of physiologic variables are measured and recorded during sleep. Physiologic sensor leads are placed on the patient in order to record brain electrical activity, eye and jaw muscle movement, leg muscle movement, airflow, respiratory effort (chest and abdominal excursion), EKG and oxygen saturation. Information is gathered from all leads and fed into a computer and outputted as a series of waveform tracings which enable the technician to visualize the various waveforms, assign a score for the test, and assist in the diagnostic process.

“Wake Time During Sleep” (WTDS), typically expressed in minutes, is the number of epochs during which the subject is awake (wake epoch) after the onset of persistent sleep and prior to final awakening, divided by two. Each epoch is defined as a 30-second duration on the PSG recording.

“Wake Time After Sleep” (WTAS), typically expressed in minutes, is the number of wake epochs after the final awakening until the end of PSG recording (i.e., a wake epoch immediately prior to the end of the recording), divided by two. If the patient does not have a wake epoch immediately prior to the end of the recording, then WTAS is zero.

“Wake Time After Sleep Onset” (WASO) is the sum of WTDS and WTAS.

“Latency to Persistent Sleep” (LPS) , typically expressed in minutes, is the number of epochs from the beginning of the PSG recording (lights-out) to the start of the first 20 consecutive epochs in which the subject is asleep (sleep epoch), divided by two.

“Total Sleep Time” (TST) , typically expressed in minutes, is the number of non-wake epochs from the beginning of the PSG recording to the end of the recording, divided by two.

“Sleep Efficiency” (SE) is the TST divided by the time in bed (8 hours), multiplied by 100 and expressed as a percentage. This also can be divided into SE for each third-of-the-night of sleep, reflecting the SE for each 160 minute time interval across the night. Finally, SE can be measured for individual hours during the night or sleep period, for example the final hour of the sleep period.

The term “fragmented sleep” can refer to interrupted sleep over a measurement period or sleep period, for example the time a patient is awake during period of measurement. Fragmentation can occur as a result of multiple awakenings or one or more awakenings of a long duration.

The term “prodrug” refers to an agent that is converted into the active drug in vivo. Prodrugs are often useful because, in some situations, they may be easier to administer than the active drug. They may, for instance, be more bioavailable by oral administration (e.g., can have improved absorption characteristics) whereas the active drug is not. The prodrug may also have improved solubility in pharmaceutical compositions over the active drug. An example, without limitation, of a prodrug would be a compound of the present invention which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety.

The term “pharmaceutically acceptable salt” refers to an ionic form of a compound that does not cause significant irritation to an organism to which it is administered and does not abrogate the biological activity and properties of the compound. Pharmaceutical salts can be obtained by reacting a compound of the invention with inorganic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutical salts can also be obtained by reacting a compound of the invention with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glutamine, tris(hydroxymethyl)methylamine, and salts with amino acids such as arginine, lysine, and the like. Pharmaceutically acceptable salts are more fully described below.

The term “low dose” can refer, for example, to a daily dose range of between about 0.1 and 6 milligrams. In some embodiments, daily dosages of low dose doxepin can be about 1, 2, 3, 4, 5, 6, 7, 8 or 9 milligrams. These dosages have reduced side effects, are surprisingly effective, and have a relatively rapid onset. In one embodiment, an initial daily dosage of about 1 milligram can be given. If the desired improvement in sleep is not achieved, then the dosage may be incrementally increased until the desired dosage is achieved or until a maximum desired dosage is reached which can be, for example, 2 milligrams, 3 milligrams, 4 milligrams, 5 milligrams, 6 milligrams, 7 milligrams, 8 milligrams or 9 milligrams. It should be noted that other dosages of doxepin can be used in the embodiments described herein. For example, the dosage can be about 0.1 to about 10 milligrams. Obtaining and Method of Making Doxepin Compounds

It is contemplated that doxepin for use in the methods described herein can be obtained from any suitable source or made by any suitable method. As mentioned, doxepin is approved and available in higher doses (75-300 milligrams) for the treatment of depression and anxiety. Doxepin HCl is available commercially and may be obtained in capsule form from a number of sources. Doxepin is marketed under the commercial name SINEQUAN® and in generic form, and can be obtained in the United States generally from pharmacies in capsule form in amounts of 10, 25, 50, 75, 100 and 150 mg dosage, and in liquid concentrate form at 10 mg/mL. Doxepin HCl can be obtained from Plantex Ltd. Chemical Industries (Hakadar Street, Industrial Zone, P.O. Box 160, Netanya 42101, Israel), Sifavitor S.p.A. (Via Livelli 1-Frazione, Mairano, Italy), or from Dipharma S.p.A. (20021 Baranzate di Bollate, Milano, Italy). Also, doxepin is commercially available from PharmacyRx (NZ) (2820 1^{st} Avenue, Castlegar, B.C., Canada) in capsule form in amounts of 10, 25, 50, 75, 100 and 150 mg. Furthermore, Doxepin HCl is available in capsule form in amounts of 10, 25, 50, 75, 100 and 150 mg and in a 10 mg/ml liquid concentrate from CVS Online Pharmacy Store (CVS.com).

Also, doxepin (11-(3-dimethylaminopropylidene) -6,11-dihydrodibenzo (b,e)oxepin) can be prepared according to the method taught in U.S. Pat. No. 3,438,981, which is incorporated herein by reference in its entirety. An example preparation is described below in Example 1. As another illustration, doxepin can be prepared from 11-[3-(Dimethylamino)propyl] -6,11- dihydrodibenzo[b,e]oxepin-11-o1 as taught in U.S. Pat. No. 3,420,851, which is incorporated herein by reference in its entirety.

As mentioned above, the methods and other embodiments described herein can utilize any suitable pharmaceutically acceptable salt or prodrug of doxepin. Therefore, the substitution or use in combination of salts and prodrugs is specifically contemplated in the embodiments described herein. The pharmaceutically acceptable salts and prodrugs can be made by any suitable method. The acids that may be used to prepare pharmaceutically acceptable acid addition salts are those that form non-toxic acid addition salts, i.e., salts containing pharmacologically acceptable anions, such as the acetate, benzenesulfonate, benzoate, bicarbonate, bisulfate, bitartrate, borate, bromide, calcium edetate, camsylate, carbonate, chloride, clavulanate, citrate, dihydrochloride, edetate, dislyate, estolate, esylate, ethylsuccinate, fumarate, gluceptate, gluconate, glutamate, glycollylarsanilate, hexylresorcinate, hydrabamine, hydrobromide, hydrochloride, iodide, isothionate, lactate, lactobionate, laurate, malate, maleate, mandelate, mesylate, methylsulfate, mucate, napsylate, nitrate, oleate, oxalate, pamoate (embonate), palmitate, pantothenate, phospate/diphosphate, polygalacturonate, salicylate, stearate, subacetate, succinate, tannate, tartrate, teoclate, tosylate, triethiodode, and valerate salts.

The term “prodrug” refers to a chemical entity that is rapidly transformed in vivo to yield an active entity of a medication, for example, doxepin, such as by hydrolysis in blood or inside tissues, for example. Examples of prodrug groups can be found in, for example, T. Higuchi and V. Stella, in “Pro-drugs as Novel Delivery Systems,” Vol. 14, A.C.S. Symposium Series, American Chemical Society (1975); H. Bundgaard, “Design of Prodrugs,” Elsevier Science, 1985; and “Bioreversible Carriers in Drug Design: Theory and Application,” edited by E. B. Roche, Pergamon Press: New York, 14-21 (1987), each of which is hereby incorporated by reference in its entirety.

### Methods of Using Low Dose Doxepin

Embodiments relate to methods for improving sleep in a patient in need thereof, without the untoward side effects of unwanted weight gain, rebound insomnia, sedative tolerance, or reduced efficacy over periods of long-term use. Some embodiments provide methods for selecting a drug to be administered to a patient in need of sleep therapy. In some embodiments the methods can include evaluating the importance of avoiding weight gain, rebound insomnia, sedative tolerance, or the need for sustained efficacy for that particular patient in conjunction with the sleep therapy; and selecting doxepin therapy for said patient if avoiding weight gain, rebound insomnia or sedative tolerance is evaluated to be of sufficient importance, or if sustained efficacy of the sleep therapy is of sufficient importance.

Also, some embodiments provide methods that include identifying a patient in need of pharmaceutical treatment of a sleep disorder while at the same time avoiding weight gain, rebound insomnia, or sedative tolerance resulting from said treatment or while having sustained or prolonged efficacy of the sleep medication over a long period of time; and treating the sleep disorder in said patient by administering doxepin, a pharmaceutically acceptable salt or prodrug of doxepin, in a dosage between about 0.5 milligram and 6 milligrams. In some embodiments, the dosage can be, for example, about 1 milligram, 3 milligrams or 6 milligrams. Thus, in one aspect, the dosage of the administered substance, such as doxepin, can be about 0.5 milligram. In one aspect, the dosage of the administered substance, for example, doxepin can be about 1 milligram. In one aspect, the dosage of the substance, for example, doxepin can be about 3 milligrams. In one aspect, the dosage of the substance, for example, doxepin can be about 6 milligrams.

Further, other embodiments relate to methods that include identifying a patient in need of a pharmaceutical treatment of a sleep disorder and also susceptible to weight gain, rebound insomnia, sedative tolerance, or reduced sleep medication efficacy over time resulting from treatment of said sleep disorder using a medication other than low-dose doxepin; and treating the sleep disorder in the patient by administering low-dose doxepin, a pharmaceutically acceptable salt or prodrug thereof in a dosage between about 0.5 milligram and 6 milligrams. In some aspects, it is contemplated to administer any other dosage as described herein.

As mentioned above and elsewhere, the methods described herein can he used to treat individuals suffering from a sleep disorder, such as insomnia. The individual can suffer from a chronic insomnia or a non-chronic insomnia. For chronic (e.g., greater than 3-4 weeks) or non-chronic insomnias, a patient may suffer from difficulties in sleep onset, sleep maintenance (interruption of sleep during the night by periods of wakefulness), sleep duration, sleep efficiency, premature early-morning awakening, or a combination thereof, Also, the insomnia may be attributable to the concurrent use of other medication, for example. The non-chronic insomnia can be, for example, a short term insomnia or a transient insomnia. The chronic or non-chronic insomnia can be a primary insomnia or an insomnia that is secondary or attributable to another condition, for example a disease such as depression or chronic fatigue syndrome. In some aspects, the patient can be one that is not suffering from an insomnia that is a component of a disease, or a patient can be treated that is otherwise healthy. As previously mentioned, the chronic or non-chronic insomnia can be a primary insomnia, that is, one that is not attributable to another mental disorder, a general medical condition, or a substance. In many cases, such conditions may be associated with a chronic insomnia and can include, but are not limited to, insomnia attributable to a diagnosable DSM-IV disorder, a disorder such as anxiety or depression, or a disturbance of the physiological sleep-wake system. In some aspects the insomnia can be non-chronic, or of short duration (e.g., less than 3-4 weeks). Examples of causes of such insomnia may be extrinsic or intrinsic and include, but are not limited to environmental sleep disorders as defined by the International Classification of Sleep Disorders (ICSD) such as inadequate sleep hygiene, altitude insomnia or adjustment sleep disorder (e.g., bereavement). Also, short-term insomnia may also be caused by disturbances such as shift-work sleep disorder.

It should be noted that in some aspects, the methods can specifically exclude one or more of any of the sleep disorders described in the previous paragraph or elsewhere herein. For example, without being limited thereto, in some aspects the methods can specifically exclude treating a chronic insomnia. As another example, without being limited thereto, in some aspects the methods can specifically exclude treating an insomnia that is attributable to a condition such as depression, anxiety or chronic fatigue.

### Preparation and Administration of Doxepin and Doxepin Compositions

In performing the methods, doxepin, a pharmaceutically acceptable salt of doxepin, or prodrug of doxepin can be administered using any suitable route or method of delivery. Doxepin, doxepin salts, and/or prodrugs can be included and administered as a composition.

Suitable routes of administration include oral, buccal, sublingual, transdermal, rectal, topical, transmucosal, or intestinal administration; parenteral delivery, including intramuscular, subcutaneous, intravenous, intramedullary injections, as well as intrathecal, direct intraventricular, intraperitoneal, intranasal, or intraocular injections.

For oral administration, the compounds can be formulated as pills, tablets, powders, granules, dragees, capsules, liquids, sprays, gels, syrups, slurries, suspensions and the like, in bulk or unit dosage forms, for oral ingestion by a patient to be treated. The compounds can be formulated readily, for example, by combining the active compound with any suitable pharmaceutically acceptable carrier or excipient.

Pharmaceutical preparations for oral use can be obtained by mixing one or more solid excipients with a pharmaceutical composition as described herein, optionally grinding the resulting mixture, and processing the mixture of granules, after adding suitable auxiliaries, if desired, to obtain tablets or dragee cores. Suitable excipients are listed below. Some examples include fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

The formulation can be in form suitable for bolus administration, for example. Oral administration can be accomplished using fast-melt formulations, for example. As a further example, the formulations can be included in pre-measured ampules or syringes, push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take any suitable form, for example, tablets or lozenges.

For topical administration, the compounds may be formulated for administration to the epidermis as ointments, gels, creams, pastes, salves, gels, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents.

For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition, any of the compounds and compositions described herein can also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt. Furthermore, any of the compounds and compositions described herein also can be formulated as a fast-melt preparation. The compounds and compositions can also be formulated and administered as a drip, a suppository, a salve, an ointment, an absorbable material such a transdermal patch, or the like.

One can also administer the compounds of the invention in sustained release forms or from sustained release drug delivery systems. A description of representative sustained release materials can be found in the incorporated materials in Remington: The Science and Practice of Pharmacy (20^{th} ed, Lippincott Williams & Wilkens Publishers (2003)), which is incorporated herein by reference in its entirety.

A variety of techniques for formulation and administration can be found in Remington. The Science and Practice of Pharmacy (20^{th} ed, Lippincott Williams & Wilkens Publishers (2003)), which is incorporated herein by reference in its entirety.

As mentioned above, the compositions and formulations disclosed herein also can include one or more pharmaceutically acceptable carrier materials or excipients. Such compositions can be prepared for storage and for subsequent administration. Acceptable carriers or diluents for therapeutic use are well known in the pharmaceutical art, and are described, for example, in the incorporated material of Remington: The Science and Practice of Pharmacy (2003). The term “carrier” material or “excipient” herein can mean any substance, not itself a therapeutic agent, used as a carrier and/or diluent and/or adjuvant, or vehicle for delivery of a therapeutic agent to a subject or added to a pharmaceutical composition to improve its handling or storage properties or to permit or facilitate formation of a dose unit of the composition into a discrete article such as a capsule or tablet suitable for oral administration. Excipients can include, by way of illustration and not limitation, diluents, disintegrants, binding agents, adhesives, wetting agents, polymers, lubricants, glidants, substances added to mask or counteract a disagreeable taste or odor, flavors, dyes, fragrances, and substances added to improve appearance of the composition. Acceptable excipients include lactose, sucrose, starch powder, maize starch or derivatives thereof, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinyl-pyrrolidone, and/or polyvinyl alcohol, saline, dextrose, mannitol, lactose, lecithin, albumin, sodium glutamate, cysteine hydrochloride, and the like. Examples of suitable excipients for soft gelatin capsules include vegetable oils, waxes, fats, semisolid and liquid polyols. Suitable excipients for the preparation of solutions and syrups include, without limitation, water, polyols, sucrose, invert sugar and glucose. Suitable excipients for injectable solutions include, without limitation, water, alcohols, polyols, glycerol, and vegetable oils. The pharmaceutical compositions can additionally include preservatives, solubilizers, stabilizers, wetting agents, emulsifiers, sweeteners, colorants, flavorings, buffers, coating agents, or antioxidants. Sterile compositions for injection can be formulated according to conventional pharmaceutical practice as described in the incorporated material in Remington: The Science and Practice of Pharmacy (2003). For example, dissolution or suspension of the active compound in a vehicle such as water or naturally occurring vegetable oil like sesame, peanut, or cottonseed oil or a synthetic fatty vehicle like ethyl oleate or the like may be desired. Buffers, preservatives, antioxidants and the like can be incorporated according to accepted pharmaceutical practice. The compounds and compositions can also be made in microencapsulated form. In addition, if desired, the injectable pharmaceutical compositions may contain minor amounts of nontoxic auxiliary substances, such as wetting agents, ph buffering agents, and the like. If desired, absorption enhancing preparations (for example, liposomes), can be utilized.

The compositions and formulations can include any other agents that provide improved transfer, delivery, tolerance, and the like. These compositions and formulations can include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol. Pharmacol. 32(2): 210-8 (2000), Charman WN “Lipids, lipophilic drugs, and oral drug delivery-some emerging concepts.” J Pharm Sci. 89(8): 967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

### Dosage

The selected dosage level can depend upon, for example, the route of administration, the severity of the condition being treated, and the condition and prior medical history of the patient being treated. However, it is within the skill of the art to start doses of the compound at levels lower than required to achieve the desired therapeutic effect and to gradually increase the dosage within an initial titration period until the desired effect is achieved with an acceptable safety profile. This type of increase in dose is not done as a result of sedative tolerance as discussed elsewhere herein. It will be understood, however, that the specific dose level for any particular patient can depend upon a variety of factors including the genetic makeup, body weight, general health, diet, time and route of administration, combination with other drugs and the particular condition being treated, and its severity. For the treatment of insomnia, preferably one dose is administered prior to bedtime.

As mentioned above, in some embodiments the preferable dosage can be between about 1 milligram and 6 milligrams. Preferably, the dosage can be about 0.5 milligrams, 1 milligram, about 2 milligrams, about 3 milligrams, about 4 milligrams, about 5 milligrams or about 6 milligrams. It should be noted that in some embodiments the dosage can be between about 0.1 milligrams and 20 milligrams or between about 0.5 milligrams and 10 milligrams. Further, the dosage can be about 7 milligrams, about 8 milligrams, about 9 milligrams, or about 10 milligrams.

### EXAMPLES

### Example 1

### Synthesis of Doxepin (11-(3-dimethylaminopropylidene)-6,11-dihydrodibenzo-[b,e]-oxepine)

Part (a) A Grignard compound was prepared in the conventional manner from 4.8 g (0.2 gram-atom) magnesium in 100 ml ether and 30 g (34 ml) (3-chloropropyl)-tertbutyl-ether and 16.40 grams (0.078 mol) 6,11-dihydrodibenzo-[b,e]-oxepine-11-one dissolved in 100 ml ether were added in dropwise fashion so that the contents of the flask boiled lightly. The mixture was heated for 1 hour with agitation in a reflux condenser to complete the reaction and then it was decomposed with ammonium chloride solution. The product which was obtained by separating, drying and eliminating the solvent produced, when the ether residue (24.0 g) was extracted with ligroin, amounted to 20.3 g (80.0% of theory) of 11-(3-tertbutoxypropyl)-11-hydroxy-6,11-dihydrodibenzo- [b,e]-oxepine, having a melting point of 124-126° C. The (3-chloropropyl)-tertbutyl ether was thereafter obtained in the following manner: 19 g (0.2 mol) 1-chloropropanol-(3), 50 ml liquid isobutylene and 0.5 ml concentrated sulfuric acid were permitted to stand for 24 hours in an autoclave, then poured into excess sodium bicarbonate solution and extracted with ether. The ether solution was dried with calcium chloride and distilled. 23.6 grams of (3-chloropropyl)-tertbutylether having a boiling point of 150-156° C. (78% of theory) were recovered.

Part (b) 30.8 grams of the 11-(3-tertbutoxypropyl)- l-hydroxy-6,11-dihydrodibenzo- [b,e]-oxepine obtained according to (a) above and 150 ml absolute alcoholic hydrochloric acid were heated for 1 hour at ebullition. After removing the solvent by evaporation, the residue was crystallized with ligroin, 21.0 grams (88.5% of theory) of 11-(3-hydroxypropylidene) -6,11-dihydrodibenzo -[b,e]-oxepine having a melting point of 108-11 1 ° C. were obtained. After recrystallization from acetic acid ester, the compound melted at 112-114° C.

Part (c) 5.0 ml thionyl chloride dissolved in 5 ml benzene were added dropwise at room temperature to 12.6 g (0.05 mol) of the 11-(3-hydroxypropylidene)-6,11-dihydrodibenzo- [b,e]-oxepine obtained in part (b) above. After 1 hour of standing, the contents of the flask were heated at ebullition for 2 hours. The volatile components were thereafter removed and the remainder distilled using high vacuum. The yield amounted to 10.6 g (78.5% of theory) of 11-(3-chloropropylidene)- 6,11-dihydrodibenzo- [b,e]-oxepine having a B.P.0.1 169-172° C., a melting point of 106-111° C. After recrystallization from 20 ml of acetic acid ester, 9.1 g (67.5% of theory) of pure product having a melting point of 113-115° C. were obtained. The crude product can however be used quite easily for further processing.

Part (d) 5.4 g (0.02 mol) of the 11-(3-chloropropylidene)-6,11-dihydrodibenzo- [b,e]-oxepine, prepared according to (c) above, in 20 ml tetrahydrofuran and 5.5 g (0.12 mol) dimethylamine in 20 ml ethanol were heated together for 3 hours using a glass autoclave and a temperature of 95-100° C. (boiling water bath). Water and 6 N hydrochloric acid were added to the contents of the autoclave and the mixture was extracted with ether. The separated, aqueous-acid components were then made alkaline with dilute caustic soda solution, and the oil thereby separated was taken up in ether. The ether residue, after distillation in a high vacuum, produced 4.1 g (73.5% of theory) of 11-(3-dimethylamino-propylidene)- 6,11-dihydrodibenzo- [b,e]-oxepine, having a B.P.0.1 147-150° C. The melting point of the hydrochloride was 182-184° C. (recrystallized from isopropanol).

### Example 2

### Phase III Study to Assess the Efficacy and Safety of Doxepin HCl in Primary Insomnia Patients with Sleep Maintenance Difficulties

A Phase III, randomized, double-blind, placebo-controlled, parallel-group, multicenter study was designed to assess the efficacy and safety of doxepin 3 milligram and 6 milligram in chronic primary insomnia patients with sleep maintenance difficulties. Patients with at least a 3-month history of primary insomnia, according to Diagnostic and Statistical Manual of Mental Disorders, Fourth Edition Text Revision (DSM-IV-TR)-defined primary insomnia were enrolled.

A total of 240 patients were planned to be enrolled, and 229 patients were randomized. Of these, 221 patients were evaluated for safety (eight patients did not receive double-blind study drug), 220 patients were included in the intent-to-treat (ITT) analysis set, and 205 patients were included in the per-protocol (PP) analysis set.

Patients were screened (Visit 1) to determine study eligibility and asked to discontinue any protocol-disallowed prescription or over-the-counter (OTC) medications used as sleep medication. At the beginning of the first week (polysomnography [PSG] Screening, Visit 2), eligible patients began two weeks of single-blind placebo dosing and had two consecutive nights of 8-hour PSG recording in the sleep center. Patients were required to meet the PSG screening criteria to remain eligible for the study. Patients were allowed to leave the sleep center during the day after the PSG Screening nights and continued to take single-blind placebo after PSG Screening (Visit 2) for five consecutive nights at home.

At the beginning of the second week of single-blind placebo dosing (Baseline, Visit 3), patients who continued to be eligible for study entry were randomly assigned to one of three treatment groups in a 1:1:1 ratio (doxepin 3 mg, 6 mg or placebo) and had two consecutive nights of 8-hour PSG recording in the sleep center. Patients were allowed to leave the sleep center during the day after the PSG nights and continued taking single-blind placebo for five consecutive nights at home.

After the PSG Screening and Baseline periods, patients began a 35-day double-blind treatment period during which they were given study drug supplies at each scheduled visit (Visits 4, 5, and 6). The scheduled study visits consisted of two consecutive nights of 8-hour PSG recording in the sleep center and next day assessments to assess sedative efficacy. Patients were asked to complete hangover/residual effects assessments, morning and evening questionnaires, and the Benzodiazepine Withdrawal Symptom Questionnaire (BWSQ) at each of these visits.

During the Discontinuation Period (Visit 7), patients received single-blind placebo and had two consecutive nights of 8-hour PSG recording in the sleep center to assess rebound insomnia. Patients were discharged from the sleep center after all final study-related procedures had been completed on the Final Study Day (Day 38, Early Termination [ET]).

Safety assessments including adverse events (AEs), vitals, and neurological assessments were performed throughout the study period. Other safety assessments, including clinical laboratory tests, electrocardiograms (ECGs), and physical examinations (PEs) were performed before and after the study treatment period. For early termination, all safety assessments, including AEs neurological assessments, clinical laboratory tests, ECGs, vitals, and PEs were performed.

### Criteria for Efficacy and Safety Evaluations:

Primary Efficacy: The primary efficacy variable was wake after sleep onset (WASO) obtained on Night 1 as determined by PSG recordings using the ITT analysis set.

Additional PSG efficacy variables: Additional efficacy variables obtained on the first PSG assessment nights (Nights 1 and 29) included WASO (Night 29), wake time during sleep (WTDS), total sleep time (TST), sleep efficiency (SE), including whole night, third of the night, and by hour, and latency to persistent sleep (LPS), latency to Stage 2 sleep, number of awakenings after sleep onset (NAASO), total wake time (TWT), wake time after sleep (WTAS), and, sleep architecture including percentage and minutes of Stage 1 , 2, and ¾ non-rapid eye movement (NREM) sleep, percentage and minutes of rapid eye movement (REM) sleep, and latency to REM sleep.

Subjective Variables: Subjective assessments included data obtained on Days 2, 16, and 30, and included subjective TST (sTST), subjective WASO (sWASO), latency to sleep onset (LSO), subjective NAASO (sNAASO), and sleep quality (all assessed by a morning questionnaire), daytime functioning (assessed by an evening questionnaire), average nightly sTST during at-home administration (5 days before the PSG visit), CGI rating scale for Severity and Therapeutic Effect completed by the clinician and patient, and a measure of insomnia severity (assessed by ISI).

Safety: Safety was assessed based on AEs, clinical laboratory tests (hematology, serum chemistry, and urinalysis), vital signs, physical examinations, neurological assessments, 12-lead ECGs, and ratings of hangover/residual effects (digit-symbol substitution test (DSST), symbol copying test (SCT), and the visual analog scale (VAS) for sleepiness), assessment of rebound insomnia using PSG recordings during the discontinuation period, and withdrawal symptoms using the benzodiazepine withdrawal symptom questionnaire (BWSQ).

Statistical Methods: Analysis of covariance (ANCOVA) methods were used to compare the mean of the WASO values obtained from PSG recordings on Night 1 between each doxepin dose (3 mg and 6 mg) and placebo, using a model that included main effects for treatment and study center, with baseline WASO as a covariate. To adjust for multiple comparisons, each pairwise comparison of doxepin to placebo was performed using Dunnett's test. Methods used to analyze other endpoints were the same as those used to analyze WASO. For LPS, latency to REM sleep, latency to Stage 2 sleep, and LSO, data were expected to be log-normally distributed and were analyzed using log-transformed values (base 10). The safety data were analyzed primarily using descriptive statistical methods.

Safety analyses included all randomized patients who received at least one dose of double-blind study medication. Treatment assignment for safety data was based on the treatment actually received.

Patients who were randomized to double-blind study medication but who never received double-blind study drug were not included in any analysis set, but were included in selected tabulations based on all randomized patients.

All efficacy analyses were performed on the Intent-to-treat (ITT) analysis set. The ITT analysis set included all randomized patients who had a corresponding PSG efficacy assessment of WASO at Visit 4, Night 1. Data were analyzed as randomized and based on observed cases.

### SUMMARY OF RESULTS

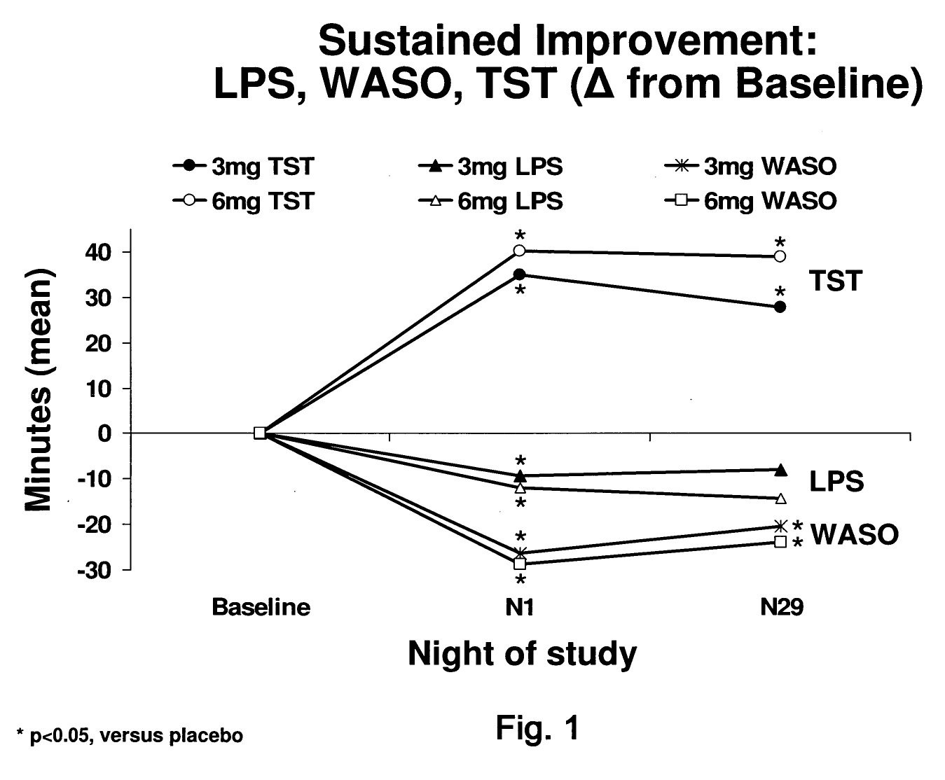

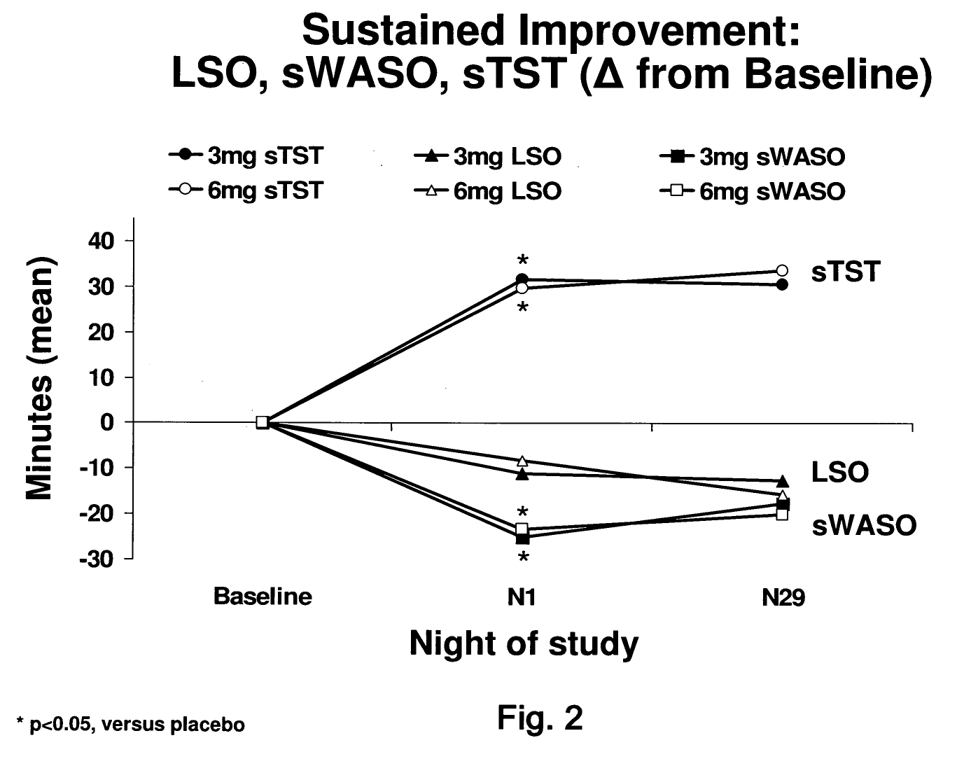

A total of 229 subjects were randomized (76 to placebo, 77 to 3 milligram, and 76 to 6 milligram). These groups were comparable with respect to weight, height, gender, and baseline sleep characteristics. A total of 203 (89%) subjects completed the study, with comparable early termination rates across treatment groups. The results are summarized in the tables below and in FIGS. 1-4.

Primary Efficacy Variable—WASO on Night 1 using the ITT Analysis Set:

On Night 1, WASO was statistically significantly decreased in the doxepin 3 mg and 6 mg groups compared with the placebo group. These improvements in WASO remained statistically significant and were sustained through the study. See Table 1.

TABLE 1
Primary Efficacy Variable: 8-Hour Wake After Sleep Onset
(Minutes) on Nights 1 and 29: Intent-to-Treat Analysis Set

| 8-hour WASO in minutes | Placebo | Doxepin 3 mg | Doxepin 6 mg |
|---|---|---|---|
| Baseline Mean (SD) | 65.6 (37.03) | 67.8 (33.56) | 65.0 (33.23) |
| Night 1 (Primary) | (n = 72) | (n = 75) | (n = 73) |
| Mean (SD) | 66.7 (50.28) | 41.4 (31.51) | 36.3 (26.14) |
| p-value | | <0.0001 | <0.0001 |
| Night 15 | (n = 69) | (n = 69) | (n = 70) |
| Mean (SD) | 60.7 (52.87) | 44.8 (27.26) | 41.9 (29.67) |
| p-value | | 0.0053 | 0.0023 |
| Night 29 | (n = 68) | (n = 68) | (n = 69) |
| Mean (SD) | 61.8 (39.71) | 47.3 (43.53) | 41.2 (37.91) |
| p-value | | 0.0299 | 0.0012 |

There were consistent statistically significant improvements for doxepin 3 mg and 6 mg compared with placebo for additional measures of sleep maintenance including TST, SE second third-of-night, TWT, and SE (final third-of-night) and WTDS (both, except for 3 mg on Night 29). In general, these positive findings for sleep maintenance were sustained through the study. See Table 3.

LPS was significantly improved in the doxepin 3 mg and 6 mg groups compared with placebo on Night 1. See Table 3.

TABLE 2
Subjective Assessment of Wake After Sleep Onset (Minutes)
on Nights 1, 15 and 29: Intent-to-Treat Analysis Set

| sWASO in minutes | Placebo | Doxepin 3 mg | Doxepin 6 mg |
|---|---|---|---|
| Baseline (Mean of | (n = 72) | (n = 75) | (n = 73) |
| Days −5 and −4) | | | |
| Mean (SD) | 74.6 (39.74) | 80.6 (48.12) | 78.2 (43.06) |
| Visit 4, Day 2 | (n = 71) | (n = 75) | (n = 73) |
| Mean (SD) | 72.5 (45.91) | 55.7 (39.81) | 54.9 (44.76) |
| P-value | | 0.0005 | 0.0007 |
| Visit 5, Day 16 | (n = 70) | (n = 69) | (n = 70) |
| Mean (SD) | 66.9 (59.65) | 59.0 (54.14) | 58.4 (49.09) |
| P-value | | 0.2582 | 0.3357 |
| Visit 6, Day 30 | (n = 68) | (n = 69) | (n = 69) |
| Mean (SD) | 59.6 (43.21) | 63.1 (47.24) | 58.2 (53.07) |
| P-value | | 0.8958 | 0.8020 |

TABLE 3
Secondary Objective 8-Hour PSG Efficacy Variables: ITT Analysis Set

| Statistic | Placebo | Doxepin 3 mg | Doxepin 6 mg |
|---|---|---|---|
| TST (minutes) | | | |
| Baseline Mean (SD) | 380.3 (44.70) | 380.3 (46.09) | 380.3 (43.09) |
| Night 1 | (n = 72) | (n = 75) | (n = 73) |
| Mean (SD) | 373.8 (72.22) | 415.3 (41.65) | 420.5 (37.07) |
| p-value | | <0.0001 | <0.0001 |
| Night 15 | (n = 69) | (n = 69) | (n = 70) |
| Mean (SD) | 389.6 (64.43) | 401.7 (48.56) | 411.3 (51.34) |
| p-value | | 0.1977 | 0.0157 |
| Night 29 | (n = 68) | (n = 68) | (n = 69) |
| Mean (SD) | 391.0 (50.50) | 408.1 (52.41) | 419.1 (44.98) |
| p-value | | 0.0262 | 0.0003 |
| SE (%) | | | |
| Baseline Mean (SD) | 79.2 (9.31) | 79.2 (9.60) | 79.2 (8.98) |
| Night 1 | (n = 72) | (n = 75) | (n = 73) |
| Mean (SD) | 77.9 (15.05) | 86.5 (8.68) | 87.6 (7.72) |
| p-value | | <0.0001 | <0.0001 |
| Night 15 | (n = 69) | (n = 69) | (n = 70) |
| Mean (SD) | 81.2 (13.42) | 83.7 (10.12) | 85.7 (10.70) |
| p-value | | 0.1977 | 0.0157 |
| Night 29 | (n = 68) | (n = 68) | (n = 69) |
| Mean (SD) | 81.5 (10.52) | 85.0 (10.92) | 87.3 (9.37) |
| p-value | | 0.0262 | 0.0003 |
| LPS (minutes) | | | |
| Baseline Mean (SD) | 38.0 (28.56) | 35.9 (29.84) | 39.1 (34.10) |
| Night 1 | (n = 72) | (n = 75) | (n = 73) |
| Mean (SD) | 45.0 (54.91) | 26.7 (23.42) | 27.1 (25.42) |
| p-value | | 0.0110 | 0.0018 |
| Night 15 | (n = 69) | (n = 69) | (n = 70) |
| Mean (SD) | 33.2 (39.75) | 38.2 (40.52) | 31.8 (36.57) |
| p-value | | 0.3644 | 0.8315 |
| Night 29 | (n = 68) | (n = 68) | (n = 69) |
| Mean (SD) | 31.3 (35.98) | 28.0 (25.99) | 24.7 (21.48) |
| p-value | | 0.9008 | 0.9989 |

TABLE 4
Subjective Assessment of Latency to Sleep Onset (Minutes)
on Nights 1, 15 and 29: Intent-to-Treat Analysis Set

| | | | Doxepin HCl |
|---|---|---|---|
| LSO in minutes | Placebo | Doxepin HCl 3 mg | 6 mg |
| Baseline (Mean of | | | |
| Days −5 and −4) | | | |
| N | 72 | 75 | 73 |
| Mean (SD) | 54.7 (30.52) | 61.4 (39.43) | 64.0 (43.45) |
| Visit 4, Day 2 | | | |
| N | 72 | 75 | 73 |
| Mean (SD) | 56.4 (46.66) | 50.3 (36.73) | 55.7 (56.96) |
| P-value | | 0.2296 | 0.0960 |
| Visit 5, Day 16 | | | |
| N | 70 | 69 | 70 |
| Mean (SD) | 55.1 (62.42) | 52.9 (35.17) | 48.7 (42.35) |
| P-value | | 0.9838 | 0.2689 |
| Visit 6, Day 30 | | | |
| N | 68 | 69 | 68 |
| Mean (SD) | 44.1 (41.26) | 48.9 (34.71) | 48.3 (47.40) |
| P-value | | 0.3567 | 0.8242 |

TABLE 5
Subjective Assessment of Total Sleep Time (Minutes) on
Nights 1, 15 and 29: Intent-to-Treat Analysis Set

| | | Doxepin HCl | Doxepin HCl |
|---|---|---|---|
| sTST in minutes | Placebo | 3 mg | 6 mg |
| Baseline (Mean | | | |
| of Days −5 and −4) | | | |
| N | 72 | 75 | 73 |
| Mean (SD) | 341.2 (52.20) | 330.0 (63.23) | 339.3 (61.45) |
| Median | 342.5 | 330.0 | 330.0 |
| Visit 4, Day 2 | | | |
| N | 72 | 75 | 73 |
| Mean (SD) | 348.3 (70.03) | 361.8 (64.03) | 369.0 (78.39) |
| P-value | | 0.0169 | 0.0256 |
| Visit 5, Day 16 | | | |
| N | 70 | 69 | 70 |
| Mean (SD) | 353.6 (85.25) | 361.0 (67.22) | 371.5 (72.06) |
| P-value | | 0.3260 | 0.1611 |
| Visit 6, Day 30 | | | |
| N | 68 | 69 | 69 |
| Mean (SD) | 365.2 (68.23) | 360.7 (68.72) | 373.0 (75.30) |
| P-value | | 0.9972 | 0.6831 |

PSG data (set forth in Tables 1, 3 and 6) and subjective data (set forth in Tables 2, 4 and 5) show improvement in each efficacy variable tested compared to baseline, with sustained results between nights 1 and 29.

As seen in Table 6 below, SE at Hour 8 was significantly improved in the doxepin 3 mg and 6 mg dose groups when compared with placebo on Night 1.

TABLE 6
Secondary Objective PSG Efficacy Variable SE -
Hour 8: ITT Analysis Set

| SE (%) - Hour 8 | Placebo | Doxepin 3 mg | Doxepin 6 mg |
|---|---|---|---|
| Baseline | (n = 72) | (n = 75) | (n = 73) |
| Mean (SD) | 78.0 (18.92) | 74.9 (22.87) | 76.4 (21.26) |
| Night 1 | (n = 72) | (n = 75) | (n = 73) |
| Mean (SD) | 74.5 (29.15) | 87.8 (14.28) | 88.4 (14.25) |
| p-value | | <0.0001 | <0.0001 |
| Night 29 | (n = 68) | (n = 68) | (n = 69) |
| Mean (SD) | 75.4 (26.06) | 81.9 (20.81) | 85.8 (19.66) |
| p-value | | 0.0524 | 0.0034 |

### No Sedative Tolerance

Surprisingly, improvements in sleep maintenance variables were, in general, sustained across study visits with no evidence of sedative tolerance using PSG recordings. The doxepin treatment demonstrated sustained efficacy during the treatment schedule. In particular, as evidenced above in Table 3, the improvement in sleep maintenance in patients treated with 3 mg and 6 mg doxepin was sustained throughout the study. Of particular note, the improvement in SE at hour 8 remained statistically significant and was sustained at night 29 for the 6 mg group. Additionally, as demonstrated in Table 1, statistically significant improvements in WASO were seen at Night 29.

### No Rebound Insomnia

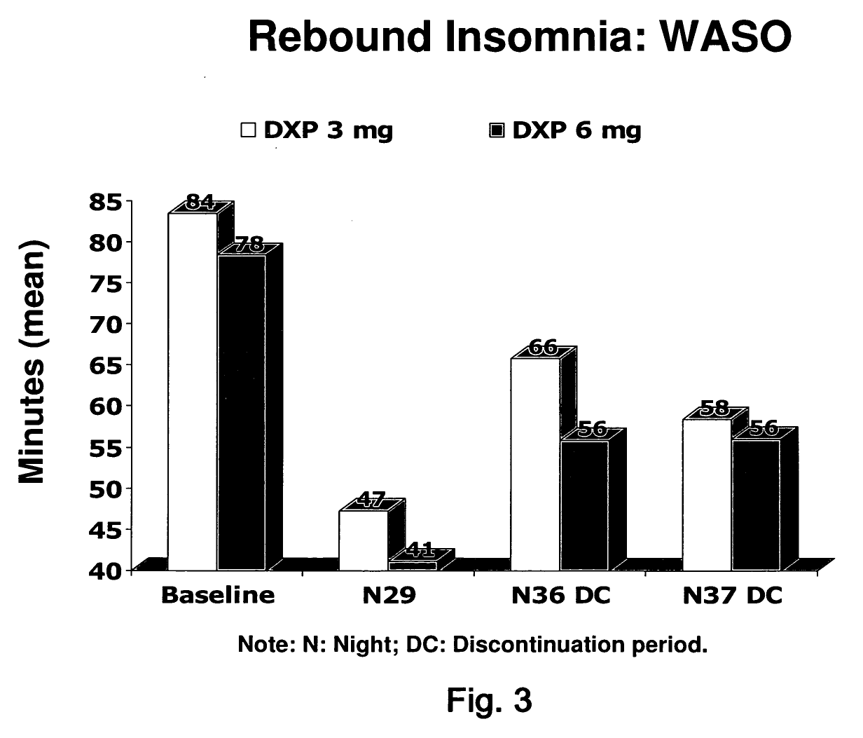

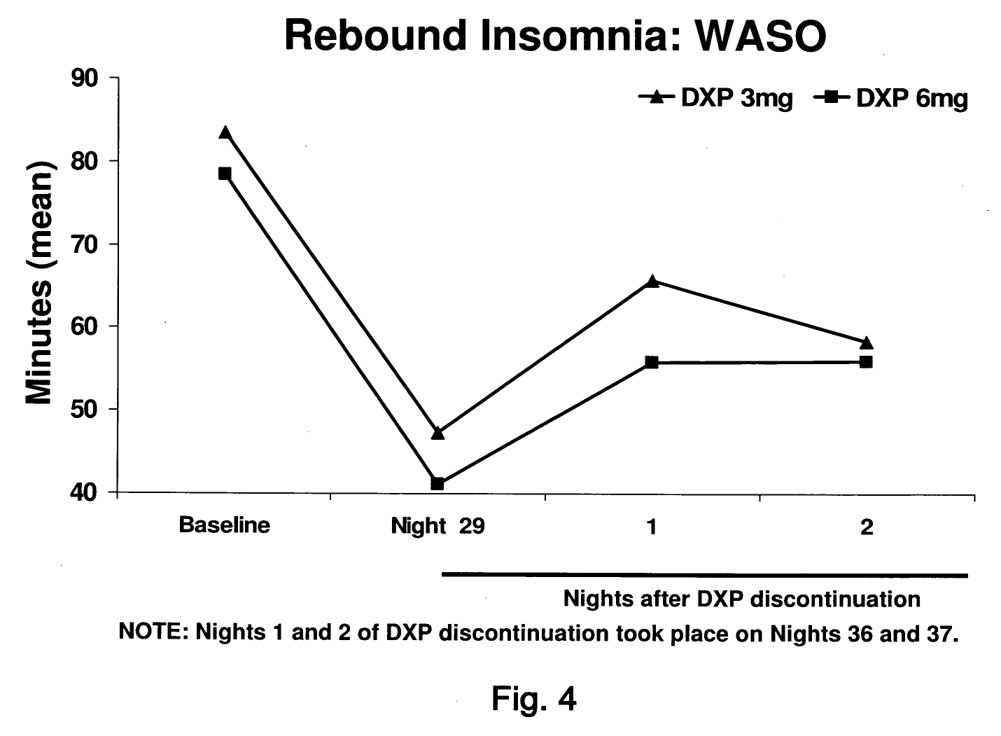

Surprisingly, as indicated in Table 7 and in FIGS. 3 and 4, this study produced no rebound insomnia (as defined by mean change in mean WASO from baseline to the first night of discontinuation; in other words, WASO measured on night 36 or 37 was not greater than the baseline measurement) during the 2-day discontinuation period following abrupt discontinuation of 35 nights of treatment. Further, as evidenced by Table 8, the frequency of incidents of rebound insomnia that were observed was surprisingly low in both the 3 mg and 6 mg doxepin groups. For example, the largest percentage of patients experiencing rebound effects was only 15% (3 mg group at night 36, as measured by mean change in WASO).

TABLE 7
Rebound Insomnia - Change in WASO Values

| | | Doxepin | Doxepin |
|---|---|---|---|
| | Placebo | HCl 3 mg | HCl 6 mg |
| WASO (minutes) | (N = 73) | (N = 75) | (N = 73) |
| Baseline* | | | |
| N | 73 | 75 | 73 |
| Mean (SD) | 81.7 (48.40) | 83.5 (41.35) | 78.4 (40.37) |
| Median | 80.0 | 79.5 | 72.5 |
| Min, Max | 7.5, 241.0 | 10.0, 204.0 | 3.0, 222.5 |
| Visit 7, Night 36 | | | |
| N | 67 | 67 | 68 |
| Mean (SD) | 58.3 (45.73) | 65.8 (48.72) | 55.9 (40.26) |
| Median | 51.0 | 50.5 | 46.0 |
| Min, Max | 3.5, 202.0 | 2.5, 183.0 | 3.5, 155.0 |
| Change from | | | |
| Baseline to | | | |
| Visit 7, Night 36 | | | |
| N | 67 | 67 | 68 |
| Mean (SD) | −21.2 (52.44) | −18.4 (48.60) | −23.6 (46.93) |
| Median | −20.5 | −22.0 | −16.5 |
| Min, Max | −208.0, 114.0 | −112.0, 124.0 | −143.0, 96.5 |
| 95% CI | (−33.97, −8.39) | (−30.28, −6.57) | (−34.99, −12.27) |
| Visit 7, Night 37 | | | |
| N | 65 | 66 | 63 |
| Mean (SD) | 56.3 (46.55) | 58.4 (42.76) | 56.0 (42.68) |
| Median | 44.5 | 48.8 | 49.0 |
| Min, Max | 4.0, 211.0 | 4.5, 181.0 | 4.5, 221.0 |
| Change from | | | |
| Baseline to | | | |
| Visit 7, Night 37 | | | |
| N | 65 | 66 | 63 |
| Mean (SD) | −20.9 (45.60) | −24.7 (43.02) | −24.3 (47.41) |
| Median | −20.5 | −20.8 | −20.0 |
| Min, Max | −175.0, 98.5 | −121.0, 73.0 | −200.5, 139.0 |
| 95% CI | (−32.20, −9.60) | (−35.26, −14.11) | (−36.26, −12.38) |

Note:
In order to be evaluable for rebound insomnia, a patient must have received placebo during the discontinuation period and have PSG data at Baseline and Night 36.
*Baseline is the worst WASO value of Nights −6 and −5 from Visit 3.

TABLE 8
Rebound Insomnia - Frequency Counts

| | | Placebo | Doxepin HCl 3 mg | Doxepin HCl 6 mg |
|---|---|---|---|---|
| PSG Parameter | Criteria | (N = 73) | (N = 75) | (N = 73) |
| | Number of Patients evaluable for rebound insomnia* | N = 67 | N = 67 | N = 68 |
| WASO | Change from Baseline** ≧35 minutes at Visit 7 | | | |
| (minutes) | | | | |
| | Night 36 | 6 (9%) | 10 (15%) | 7 (10%) |
| | Night 36 and Night 37 | 1 (1%) | 1 (1%) | 3 (4%) |
| LPS (minutes) | Change from Baseline** ≧20 minutes at Visit 7 | | | |
| | Night 36 | 4 (6%) | 7 (10%) | 8 (12%) |
| | Night 36 and Night 37 | 0 (0%) | 3 (4%) | 2 (3%) |
| TST (minutes) | Change from Baseline** ≦−30 minutes at Visit 7 | | | |
| | Night 36 | 5 (7%) | 9 (13%) | 6 (9%) |
| | Night 36 and Night 37 | 0 (0%) | 3 (4%) | 2 (3%) |

*In order to be evaluable for rebound insomnia, a patient must have received placebo during the discontinuation period and have PSG data at Baseline and Night 36. Percentages are based on the number of patients evaluable for rebound insomnia.
**Baseline is the worst PSG parameter value of Nights −6 and −5 from Visit 3.

### No Weight Gain

Surprisingly, there were no clinically relevant changes noted relative to placebo in patient weight throughout the study. As shown in Table 9, none of the patients in the 3 mg or 6 mg doxepin groups experienced a weight increase of at least 7%. Additionally, there were no clinically relevant changes noted in other clinical laboratory tests, vital sign measurements, physical examinations, neurological assessments, or ECG parameters, and no patient discontinued treatment due to an abnormal ECO parameter, vital sign measurement, laboratory test, physical examination, or neurological assessment.

TABLE 9
Weight Gain

| | | | Doxepin HCl | Doxepin HCl |
|---|---|---|---|---|
| | | Placebo | 3 mg | 6 mg |
| Parameter | Criteria | (N = 73) | (N = 75) | (N = 73) |
| Body Weight | Change from | −0.3 (2.46) | 0.3 (2.21) | 0.0 (2.19) |
| | Screening, | | | |
| | mean (±SD) kg | | | |
| | Subjects with | 1/67 (1%) | 0/74 (0%) | 0/70 (0%) |
| | ≧7% increase | | | |
| | Subjects with | 1/67 (1%) | 1/74 (1%) | 1/70 (1%) |
| | ≧7% decrease | | | |

### Conclusions

The safety and efficacy of doxepin 3 mg and 6 mg compared with placebo was demonstrated in adult patients with at least a 3-month history of primary insomnia with sleep maintenance difficulties.

In summary, doxepin at 3 mg and 6 mg demonstrated efficacy on sleep onset, sleep maintenance, and sleep duration into the final hour of the night. The doxepin treatment showed no significant next day residual effects, no withdrawal effects, no rebound insomnia upon abrupt discontinuation of dosing, no weight gain, and no sedative tolerance. The doxepin treatment showed sustained efficacy over a period of long-term use.

Many modifications and variations of the embodiments described herein may be without departing from the scope, as is apparent to those skilled in the art. The specific embodiments described herein are offered by way of example only.

We claim:

1. A method of providing a sleep therapy, comprising:
informing a medical caregiver or a patient that doxepin in an amount of about 1 to 6 mg does not result in significant weight gain; and
providing the caregiver or the patient with about 1-6 mg of doxepin for consumption by the patient.

2. A method of providing a sleep therapy, comprising:
informing a medical caregiver or a patient that doxepin in an amount of about 1 to 6 mg does not result in sedative tolerance or that the doxepin in said amount maintains sustained efficacy as a sleep therapeutic over a period of long term use; and
providing the caregiver or the patient with about 1-6 mg of doxepin for consumption by the patient.

3. The method of claim 2, wherein the patient is suffering from chronic insomnia.

4. A method of providing a sleep therapy, comprising:
informing a medical caregiver or a patient that doxepin in an amount of about 1 to 6 mg does not result in rebound insomnia; and
providing the caregiver or the patient with about 1-6 mg of doxepin for consumption by the patient.

5. The method of claim 4, wherein the patient is suffering from transient insomnia.

6. The method of any of claims 1, 2 and 4, wherein the doxepin is provided in an amount of about 1 milligram.

7. The method of any of claims 1, 2 and 4, wherein the doxepin is provided in an amount of about 3 milligrams.

8. The method of any of claims 1, 2 and 4, wherein the doxepin is provided in an amount of about 6 milligrams.

9. A method, comprising:
identifying a patient in need of pharmaceutical treatment of a sleep disorder and also susceptible to one or more of weight gain resulting from treatment of said sleep disorder using a medication other than doxepin, sedative tolerance resulting from treatment of said sleep disorder using a medication other than doxepin, rebound insomnia resulting from treatment of said sleep disorder using a medication other than doxepin, or reduced sleep medication efficacy resulting from treatment of said sleep disorder using a medication other than doxepin; and
treating said sleep disorder in said patient by administering doxepin, a pharmaceutically acceptable salt or prodrug thereof in a dosage between about 0.5 milligram and 6 milligrams.

10. The method of claim 9, wherein the dosage of doxepin is about 1 milligram.

11. The method of claim 9, wherein the dosage of doxepin is about 3 milligrams.

12. The method of claim 9, wherein the dosage of doxepin is about 6 milligrams.

13. A method for selecting a drug to be administered to a patient in need of sleep therapy, comprising:
evaluating the importance of avoiding one or more of weight gain, rebound insomnia, sedative tolerance, or a lack of sustained efficacy during chronic use for that particular patient in conjunction with said sleep therapy; and
selecting doxepin therapy for said patient if avoiding the one or more of weight gain, rebound insomnia, sedative tolerance, or a lack of sustained efficacy during chronic use is evaluated to be of sufficient importance.

14. The method of claim 13, further comprising providing a daily dosage of doxepin, a doxepin prodrug or a pharmaceutically acceptable salt of doxepin in an amount of about 0.5 milligrams to about 6 milligrams.

15. The method of any of claims 9 and 13, wherein the patient is in need of treatment for transient insomnia or short term insomnia.

16. The method of any of claims 9 and 13, wherein the patient is in need of treatment for chronic insomnia.
